(19) United States
(12) Patent Application Publication
(10) Pub. No.: US 2018/0358561 A1
(43) Pub. Date: Dec. 13, 2018

(54) ORGANIC ELECTRON TRANSPORT MATERIAL

(71) Applicant: GUANGDONG AGLAIA OPTOELECTRONIC MATERIALS CO. LTD, Foshan, Guangdong (CN)

(72) Inventors: Kam-Hung LOW, Foshan (CN); Chin-Hsin CHEN, Foshan (CN); Zhe LI, Foshan (CN); Lei DAI, Beijing (CN); Lifei CAI, Beijing (CN)

(73) Assignee: GUANGDONG AGLAIA OPTOELECTRONIC MATERIALS CO. LTD, Foshan, Guangdong (CN)

(21) Appl. No.: 16/060,939

(22) PCT Filed: Nov. 30, 2016
(86) PCT No.: PCT/CN2016/107854

§ 371 Date: Jun. 9, 2018

(30) Foreign Application Priority Data
Dec. 9, 2015 (CN) 201510902765.9

(51) Int. Cl. H01L 51/00; C07C 13/66

(52) U.S. Cl. CPC H01L 51/0058; H01L 51/5072; C07C 13/66; H01L 51/0054

(57) ABSTRACT

The present invention relates to an organic electron transport material having a compound of the structure shown in formula (I), wherein R1-R4 independently represent hydrogen, C1-C8 substituted or substituted alkyl, C2-C8 substituted or unsubstituted alkenyl, or C2-C8 substituted or unsubstituted alkynyl, the substituents being C1-C4 alkyl or halogen. Device experiments show that an electronic-only organic semiconductor diode device and an organic electroluminescent device manufactured by the organic electron transport material of the present invention have good electron transport performance, high and stable luminance, and a long device life.

### TECHNICAL FIELD

The present invention relates to a novel organic electron transport material, which is formed into a thin film by vacuum deposition and may be applied to an electronic-only organic semiconductor diode device.

### BACKGROUND ART

An electronic-only organic semiconductor diode device is one type of single-carrier devices and is used as a power semiconductor device for a switch or a rectifier of a smart digital power integrated circuit. The electron transport material of the present invention can also be applied to organic electroluminescent devices and field effect transistors.

The electronic-only organic semiconductor diode device is a device that is manufactured by spin-coating or depositing one or more layers of organic materials between two electrodes made of metal, inorganic matters or organic compounds. A classical single-layer electronic-only organic semiconductor diode device includes an anode, an electron transport layer, and a cathode. A hole barrier layer may be added between an anode and an electron transport layer of a multi-layer electronic-only organic semiconductor diode device, and an electron injection layer may be added between the electron transport layer and a cathode. The hole barrier layer, the electron transport layer and the electron injection layer are composed of a hole barrier material, an electron transport material and an electron injecting material, respectively. After a voltage connected to the electronic-only organic semiconductor diode device reaches a turn-on voltage, electrons generated by the cathode are transported through the electron transport layer to the anode, and conversely, holes cannot be injected from the anode. The electron transport material in the electronic-only organic semiconductor diode device can be applied to other semiconductor devices such as an organic electroluminescent device. The organic electroluminescent device has a huge market, so the stable and efficient organic electron transport material plays an important role in the application and promotion of organic electroluminescent devices, and is also an urgent need for the application and promotion of organic electroluminescent large-area panel display.

Existing electron transport materials bathophenanthroline (BPhen) and bathocuproine (BCP) which are frequently used in the market can basically meet the market demand of organic electroluminescent panels, but their efficiency and stability still need to be further improved. The BPhen and BCP materials have the disadvantage of easy crystallization. Once the electron transport material crystallizes, a charge transfer mechanism among molecules is different from an amorphous film mechanism that operates normally, resulting in a change in the electron transport properties. When the electron transport material is used in the organic electroluminescent device, the electrical conductivity of the entire device will change after a period of time, causing electron and hole charge mobility to become unbalanced, resulting in decrease of performance of the device and local short-circuiting possibly occurring in the device, and thereby affecting the stability of the device, and even resulting in failure of the device (Reference document: Journal of Applied Physics 80, 2883 (1996); doi: 10.1063/1.363140).

Although the synthesis process of BPhen has been quite mature (reference document: WO 2010127574 A1), a raw material o-phenylenediamine (CAS 95-54-5) used therein has been listed as a highly toxic compound to aquatic organisms by the US Environmental Protection Agency. In consideration of protection of environment and water resources in China against pollution, the demand for research and development of a novel electron transport material is very urgent. A non-heterocyclic fluoranthene compound contains only carbon and hydrogen and can be used as an electron transport material and a luminescent material in an organic light emitting diode (OLED) device (reference document: WO 2013182046 A1), but its transport efficiency and thermal stability still need to be further improved.

### SUMMARY OF THE INVENTION

In view of the defects of the above materials, the present invention provides an organic electron transport material that has high morphological stability and may be applied to a long-life electronic-only organic semiconductor diode device and an organic electroluminescent device, and has good electron transport performance and high luminance.

An organic electron transport material has a compound of a structure shown in formula (I),

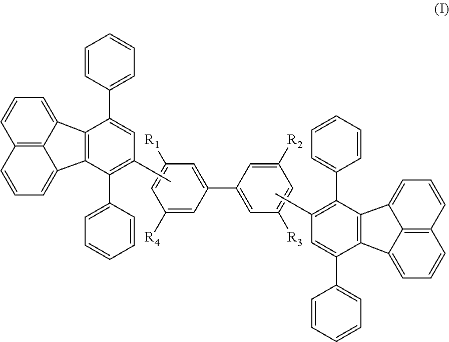

wherein R1-R4 independently represent hydrogen, C1-C8 substituted or substituted alkyl, C2-C8 substituted or unsubstituted alkenyl, or C2-C8 substituted or unsubstituted alkynyl, the substituents being C1-C4 alkyl or halogen.

Preferably, R1-R4 independently represent hydrogen, C1-C4 substituted or substituted alkyl, C2-C4 substituted or unsubstituted alkenyl, or C2-C4 substituted or unsubstituted alkynyl.

Preferably, R1-R4 independently represent hydrogen, or C1-C4 alkyl.

Preferably, R1-R4 are identical.

Preferably, R1-R4 preferably represent hydrogen.

The compound shown in formula (I) is the following compound having the structure:

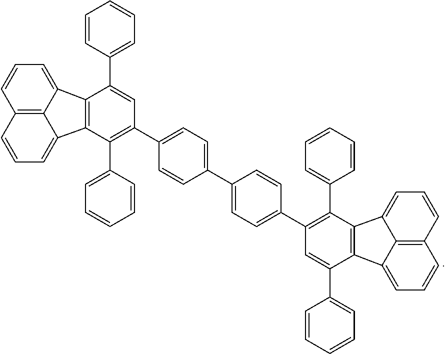 [SMILES: c1ccc(-c2cc(-c3ccc(-c4ccc(-c5cc(-c6ccccc6)c6c(c5-c5ccccc5)-c5cccc7cccc-6c57)cc4)cc3)c(-c3ccccc3)c3c2-c2cccc4cccc-3c24)cc1]

The organic layer is one or more of a hole barrier layer, an electron transport layer, and an electron injection layer. It should be pointed out in particular that these organic layers mentioned above can be present as required, rather than every layer being present.

The hole barrier layer, the electron transport layer and/or the electron injection layer contain the compound of the formula (I).

The compound of formula (I) is an electron transport material.

The total thickness of the organic layers of the electronic device of the present invention is 1 nm to 1000 nm, preferably 1 nm to 500 nm, and more preferably 5 nm to 300 nm.

The organic layer may be formed into a thin film by evaporation or spin coating.

As mentioned above, the compounds of formula (I) of the present invention are as follows, but are not limited to the structures listed:

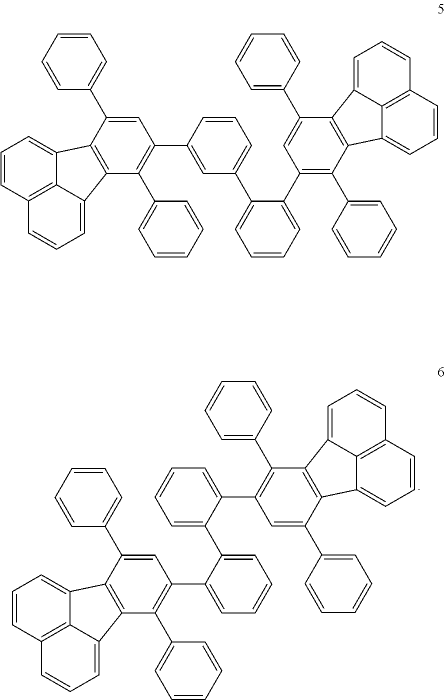

Device experiments show that the electronic-only organic semiconductor diode device and the organic electroluminescent device manufactured by the organic electron transport material of the present invention have good electron transport performance, high and stable luminance, and a long device life.

### BRIEF DESCRIPTION OF THE DRAWINGS

FIG. 1 is an HPLC diagram of a compound 1;

FIG. 2 is a carbon spectrogram of the compound 1;

FIG. 3 is a thermogravimetry—TGA diagram of the compound 1;

FIG. 4 is a structural diagram of an electronic-only organic semiconductor diode device according to the present invention, wherein 10 represents a glass substrate, 20 represents an anode, 30 a hole barrier layer, 40 an electron transport layer, 50 an electron injection layer, and 60 a cathode;

FIG. 5 is a voltage-current density diagram of a device 1 of the present invention;

FIG. 6 is a voltage-current density diagram of a device 2 of the present invention;

FIG. 7 is a voltage-current density diagram of devices 3 and 4 of the present invention;

FIG. 8 is a current density-current efficiency diagram of the devices 3 and 4 of the present invention;

FIG. 9 is a luminance-color coordinate y diagram of the devices 3 and 4 of the present invention;

FIG. 10 is an emission spectrum diagram of the devices 3 and 4 of the present invention; and

FIG. 11 is a structural diagram of an organic electroluminescent device according to the present invention, wherein 10 represents a glass substrate, 20 represents an anode, 30 a hole injection layer, 40 a hole transport layer, 50 a light emitting layer, 60 an electron transport layer, 70 a cathode.

### DETAILED DESCRIPTIONS OF THE PREFERRED EMBODIMENTS

In order to describe the present invention in more detail, the following examples are given, but the present invention is not limited to these.

### Example 1

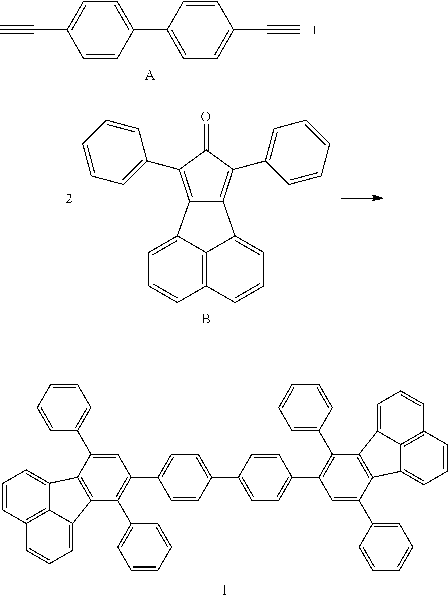

### Synthesis of Compound 1

A compound A is synthesized according to the reference document: ACS Macro Letter, 2014, Processes 3, and 10-15. A compound B is synthesized according to the process of reference document: WO 2013182046 A1.

Reaction delivery: sequentially adding the compound A (2.21 g, 11 mmol), the compound B (7.80 g, 22 mmol), and diphenyl ether (100 mL) to a 250-mL reaction flask; after evacuating hydrogen three times, heating to 260° C., and preserving the heat and reacting for 8 hours till the compound B completely reacts under TLC and HPLC detection, the color of the reaction solution changing from black to yellow during the reaction.

Treatment after reaction: stopping heating and cooling to 20° C.; adding methanol (100 mL) and stirring for 2 h to separate solid out; washing a filter cake with methanol and drying in vacuum to obtain a crude product; adding ethyl acetate to the crude product and pulping to obtain a yellow compound 1 (4.32 g, yield 46%, HPLC purity 99.0%); performing vacuum sublimation (360° C., 2×10⁻⁵ torr, 8h) to obtain light yellow solid powder with a purity of 99.5%.

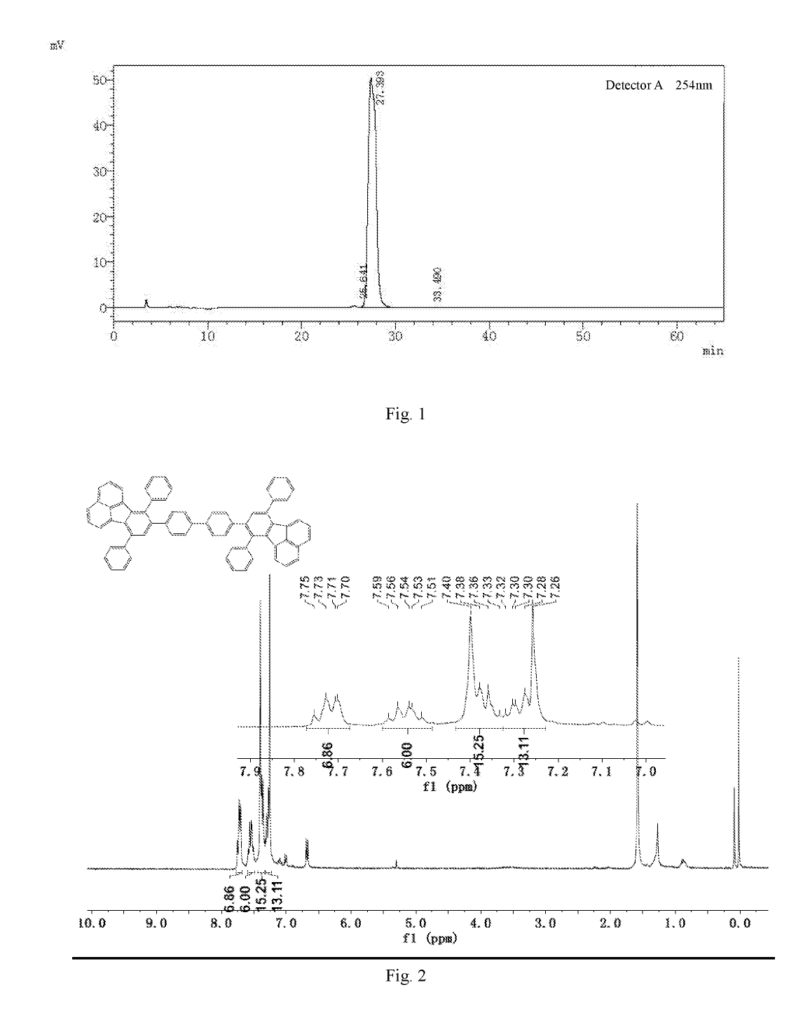

See FIG. 1, the liquid phase conditions are as follows:

chromatographic column: Inertsil ODS-SP 4.6*250 mm, 5 μm, column temperature: 40° C.;

solvent: DCM, moving phase: ACN, detection wavelength: 254 nm.

The peak calculation chart is as follows:

“Peak Table”
Detector A 254 nm

| | | Retention | | | |
|---|---|---|---|---|---|
| Peak No. | Compound | Time | Height | Area | Area % |
| 1 | | 25.641 | 228 | 11458 | 0.386 |
| 2 | Product | 27.393 | 50885 | 2955536 | 99.576 |
| | Y15050703-01 | | | | |
| 3 | | 33.490 | 14 | 1124 | 0.038 |
| Total | | | 51127 | 2968119 | 100.000 |

¹H NMR (300 MHz, CDCl3) δ 7.78-7.66 (m, 8H), 7.59-7.46 (m, 6H), 7.43-7.33 (m, 116H), 7.32-7.46 (m, 12H). See FIG. 2.

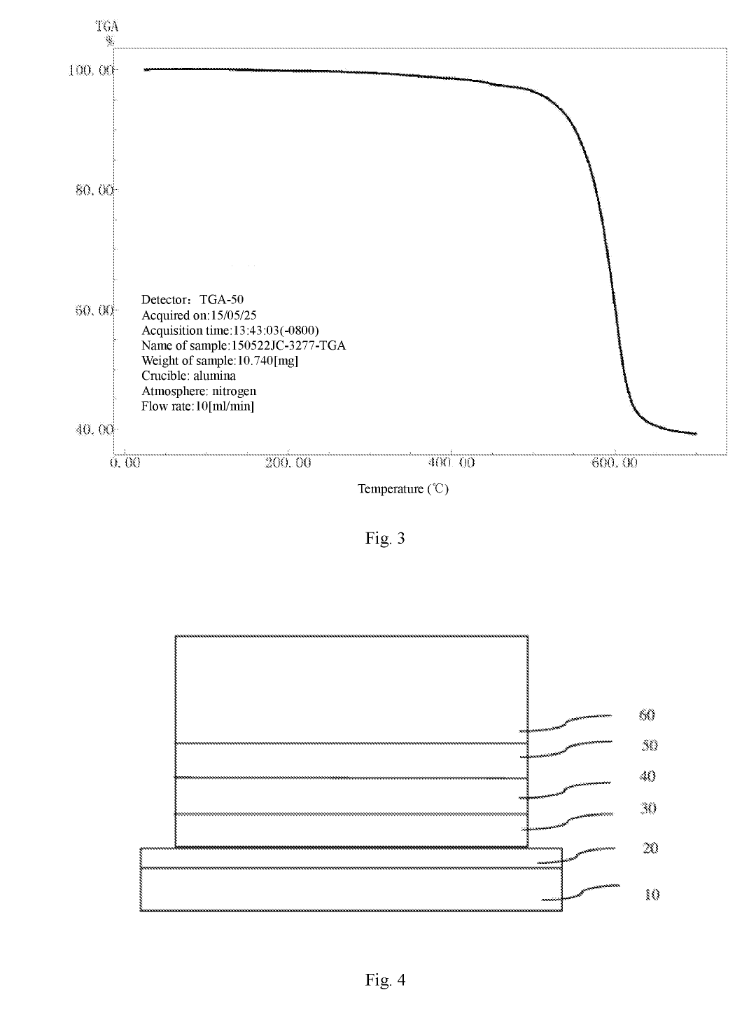

The TGA diagram is shown in FIG. 3.

### Example 2

### Preparation of Electronic-Only Organic Semiconductor Diode Device 1

The electronic-only organic semiconductor diode device is manufactured by an organic electron transport material of the present invention.

First, a transparent conductive ITO glass substrate 10 (with an anode 20 on the top) is sequentially washed with a detergent solution and deionized water, ethanol, acetone and deionized water, and then subject to oxygen plasma treatment for 30 seconds.

Then, BCP which is 5 nm thick is evaporated on ITO as a hole barrier layer 30.

Then, a compound 1 which is 100 nm thick is evaporated on the hole injection layer as an electron transport layer 40.

Then, lithium fluoride which is 1 nm thick is evaporated on the electron transport layer as an electron injection layer 50.

At last, aluminum which is 100 nm thick is evaporated on the electron injection layer as a device cathode 60.

The structural diagram is as shown in FIG. 4.

By using the space charge limited current (SCLC), the relationship between the current density and the electric field intensity is as shown in equation (1):

$$J = \frac{9}{8} ɛ {ɛ}_{0} \frac{{E}^{2}}{L} {μ}_{0} exp ( β \sqrt{E} ) \qquad ( 1 )$$

wherein, J is a current density (mA cm ⁻²), ε is a relative dielectric constant (it is generally 3 in an organic material), ε₀ is a vacuum dielectric constant (8.85×10^{—14} C V ⁻¹ cm⁻¹), E is an electric field intensity (V cm⁻¹), L is a thickness (cm) of a sample in the device, to is an electric charge mobility (cm² V⁻¹ s⁻¹) under zero electric field, and β is a Poole-Frenkel factor which indicates how fast the mobility changes with the electric field intensity.

The structural formula in the device is as follows:

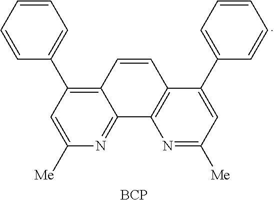

### Comparative Example 1

### Preparation of Electronic-Only Organic Semiconductor Diode Device 2

The method is the same as that of example 2, but the common commercially available compound TmPyPB is used as the electron transport layer 40 to manufacture a comparative electronic-only organic semiconductor diode device.

The structural formula in the device is as follows:

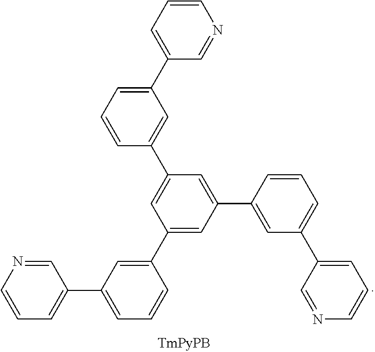 [SMILES: c1cncc(-c2cccc(-c3cc(-c4cccc(-c5cccnc5)c4)cc(-c4cccc(-c5cccnc5)c4)c3)c2)c1]

### Electron Mobility (cm² V⁻¹ s⁻¹) of the Manufactured Device

| | | Electron | Electron | Electron |
|---|---|---|---|---|
| | | Mobility | Mobility | Mobility |
| | | 1 × 10⁵ | 5 × 10⁵ | 1 × 10⁶ |
| | | V/cm Under | V/cm Under | V/cm Under |
| Device | | Operating | Operating | Operating |
| No. | μo | Electric Field | Electric Field | Electric Field |
| 1 | 4.74 × 10⁻¹¹ | 1.28 × 10⁻⁹ | 7.51 × 10⁻⁸ | 1.59 × 10⁻⁶ |
| 2 | 5.12 × 10⁻¹³ | 5.81 × 10⁻¹¹ | 2.01 × 10⁻⁸ | 1.61 × 10⁻⁶ |

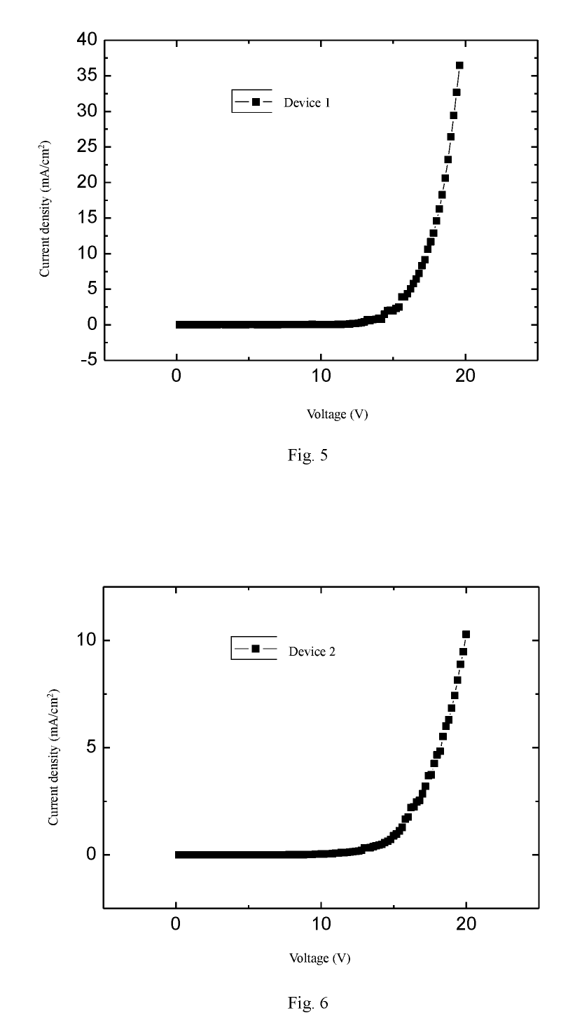

The electron mobility of the device 1 and the electron mobility of the device 2 under operating electric fields of 1×10⁵ V/cm and 5×10⁵ V/cm are calculated according to formula (1) and data in FIGS. 5 and 6. As can be seen from the results, under operating electric fields of 1×10⁵ V/cm and 5×10⁵ V/cm, the electron mobility of the device 1 is higher than the electron mobility of the device 2; the electron mobility of the device 1 and the electron mobility of the device 2 are almost the same under the operating electric field of 1×10⁶ V/cm, which indicates that the compound 1 has better electron transport property.

### Example 3

### Preparation of Organic Electroluminescent Device 3

OLED is manufactured by the organic electronic material of the present invention.

First, a transparent conductive ITO glass substrate 10 (with an anode 20 on the top) is sequentially washed with a detergent solution and deionized water, ethanol, acetone and deionized water, and then subject to oxygen plasma treatment for 30 seconds.

Then, a compound C which is 90 nm thick is evaporated on ITO as a hole injection layer 30.

Then, a compound D is evaporated to form a hole transport layer 40 which is 30 nm thick.

Then, a compound E (2%) and a compound F (98%) which are 40 nm thick are evaporated on the hole transport layer as a light emitting layer 50.

Then, the compound 1 (50%) and LiQ (50%) which are 40 nm thick are evaporated on the light emitting layer as an electron transport layer 60.

At last, Al which is 100 nm thick is taken as a device cathode 70.

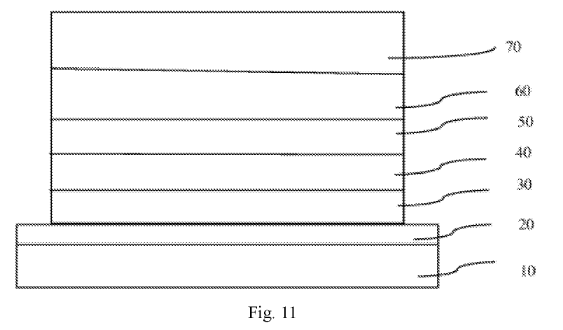

(The structure diagram is as shown in FIG. 11)

### Example 4

### Preparation of Organic Electroluminescent Device 4

OLED is manufactured by commercially available materials.

First, a transparent conductive ITO glass substrate 10 (with an anode 20 on the top) is sequentially washed with a detergent solution and deionized water, ethanol, acetone and deionized water, and then subject to oxygen plasma treatment for 30 seconds.

Then, a compound C which is 90 nm thick is evaporated on ITO as a hole injection layer 30.

Then, a compound D is evaporated to form a hole transport layer 40 which is 30 nm thick.

Then, a compound E (2%) and a compound F (98%) which are 40 nm thick are evaporated on the hole transport layer as a light emitting layer 50.

Then, the compound G (50%) and LiQ (50%) which are 40 nm thick are evaporated on the light emitting layer as an electron transport layer 60.

At last, Al which is 100 nm thick is taken as a device cathode 70.

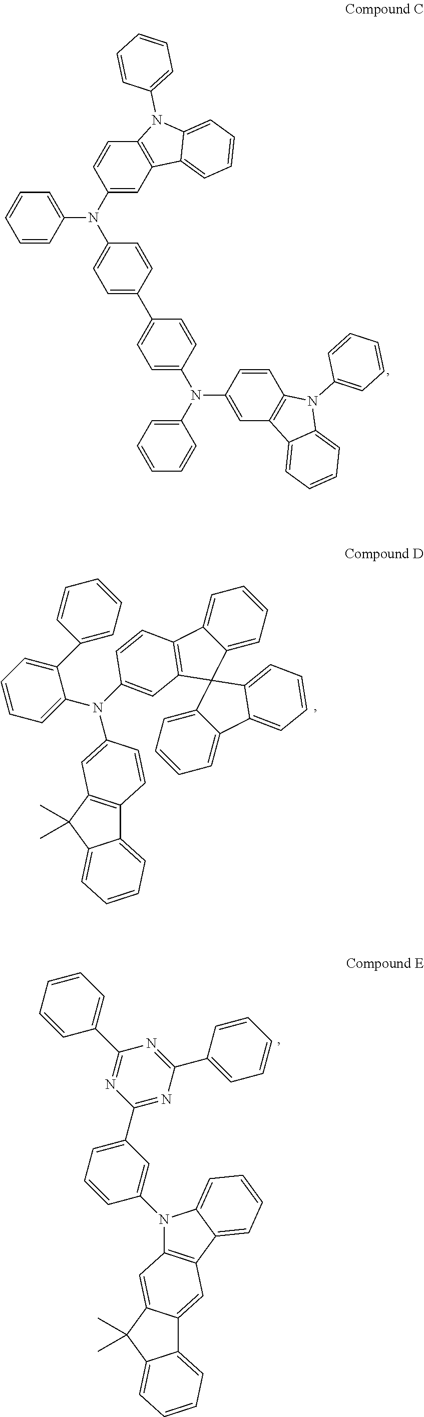 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2ccc(N(c3ccc4c(c3)C3(c5ccccc5-c5ccccc53)c3ccccc3-4)c3ccccc3-c3ccccc3)cc21 ; CC1(C)c2ccccc2-c2cc3c4ccccc4n(-c4cccc(-c5nc(-c6ccccc6)nc(-c6ccccc6)n5)c4)c3cc21]

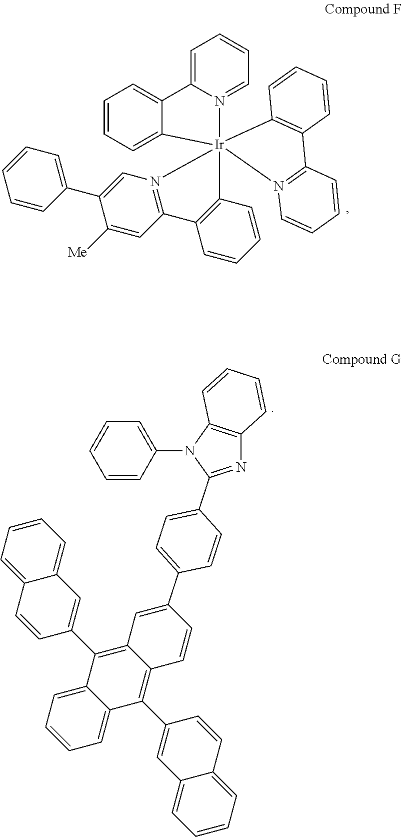

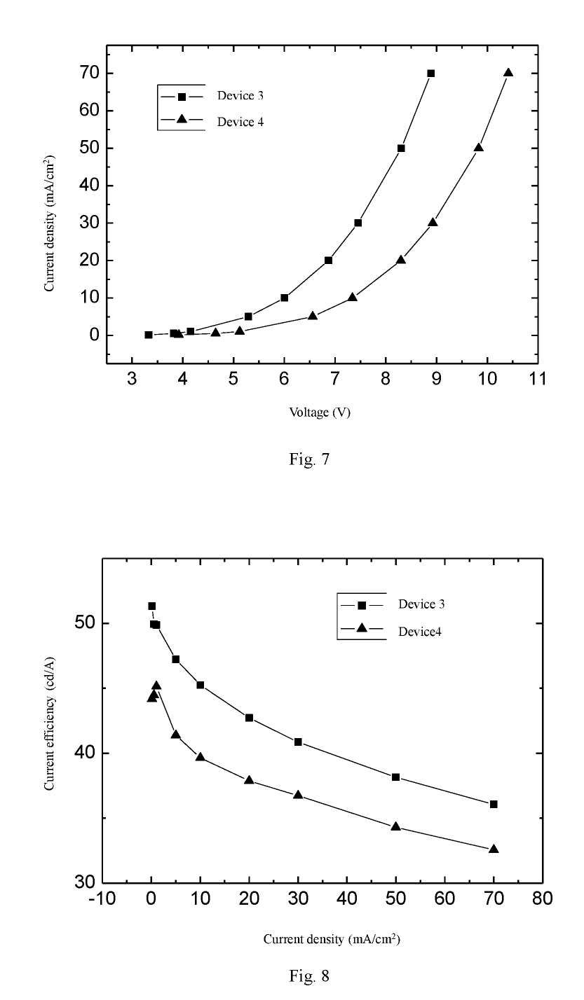

As can be seen from FIGS. 7-8 and from the comparison of the device 3 and the device 4, the electron transport performance of the compound 1 is superior to that of the comparative commercially available compound G

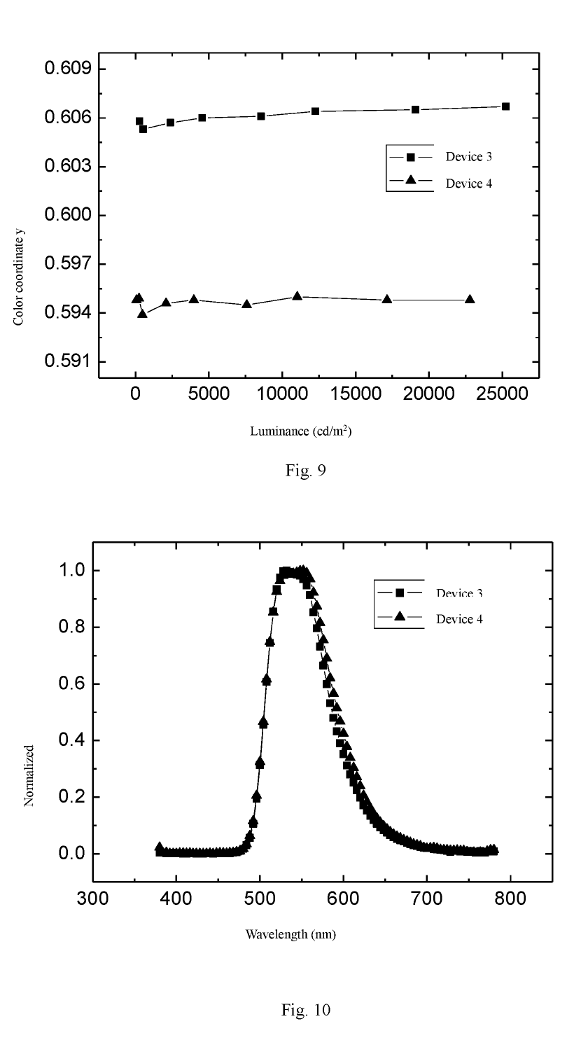

The followings can be calculated from FIGS. 9-10:

the manufactured device 3, under an operating current density of 20 mA/cm², has a luminance of 7584 cd/m², a current efficiency up to 37.9 cd/A, EQE of 11.1 under 14.3 lm/W, as well as a CIEx of 0.3709 and a CIEy of 0.5945 of green light emission.

the manufactured device 4, under an operating current density of 20 mA/cm², has a luminance of 8555 cd/m², a current efficiency up to 42.7 cd/A, EQE of 12.4 under 19.5 lm/W, as well as a CIEx of 0.3578 and a CIEy of 0.6061 of green light emission.

1. An organic electron transport material having a compound of a structure shown in formula (I) 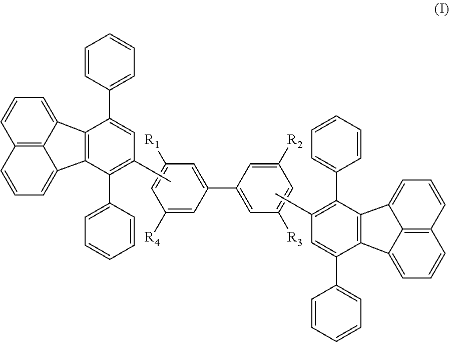
wherein R1-R4 independently represent hydrogen, C1-C8 substituted or substituted alkyl, C2-C8 substituted or unsubstituted alkenyl, or C2-C8 substituted or unsubstituted alkynyl, the substituents being C1-C4 alkyl or halogen.

2. The organic electron transport material according to claim 1, wherein R1-R4 independently represent hydrogen, C1-C4 substituted or substituted alkyl, C2-C4 substituted or unsubstituted alkenyl, or C2-C4 substituted or unsubstituted alkynyl.

3. The organic electron transport material according to claim 2, wherein R1-R4 independently represent hydrogen, or C1-C4 alkyl.

4. The organic electron transport material according to claim 3, wherein R1-R4 are identical.

5. The organic electron transport material according to claim 1, R1-R4 preferably represent hydrogen.

6. The organic electron transport material according to claim 1, having a compound of a structure shown in the following formulas:

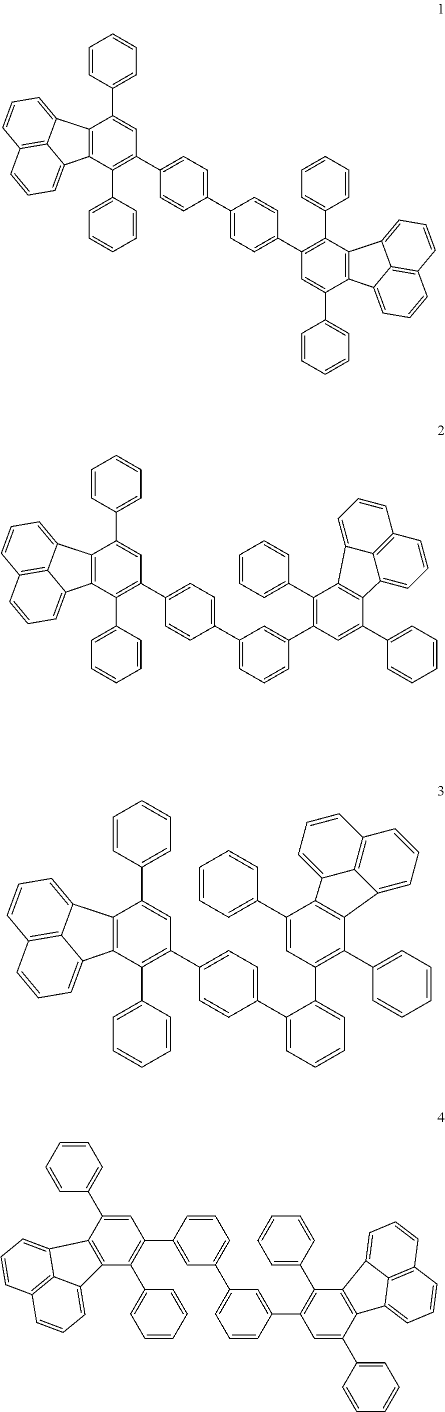

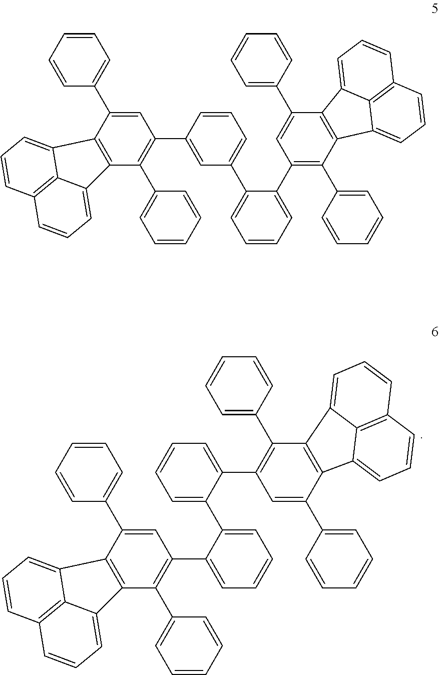

7. The organic electron transport material according to claim 1, having a compound of a structure shown in the following formula:

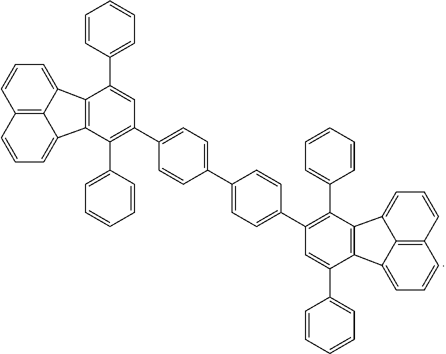 [SMILES: c1ccc(-c2cc(-c3ccc(-c4ccc(-c5cc(-c6ccccc6)c6c(c5-c5ccccc5)-c5cccc7cccc-6c57)cc4)cc3)c(-c3ccccc3)c3c2-c2cccc4cccc-3c24)cc1]

8. An application of the organic electron transport material of any one of claims 1 to 7 in an electronic-only organic semiconductor diode device and an organic electroluminescent device.
